(19) United States
(12) Patent Application Publication
(10) Pub. No.: US 2018/0113113 A1
(43) Pub. Date: Apr. 26, 2018

(54) SYSTEM FOR DEALING WITH THE LIBERALIZATION OF RECREATIONAL DRUGS

(71) Applicant: Alcohol countermeasure Systems (International) Inc., Toronto (CA)

(72) Inventors: Felix J.E. Comeau, Toronto (CA); Bruce A. Bailey, Toronto (CA); Steven H. Leach, Toronto (CA); Matt Goledzinowski, Toronto (CA)

(21) Appl. No.: 15/790,665

(22) Filed: Oct. 23, 2017

Related U.S. Application Data
(60) Provisional application No. 62/411,759, filed on Oct. 24, 2016.
(60) Provisional application No. 62/442,081, filed on Jan. 4, 2017.

(51) Int. Cl. G01N 33/497; G01N 35/00; G07B 1/08; G07C 1/14; G06K 19/10

(52) U.S. Cl. CPC G01N 33/4972; G01N 35/00732; G07B 1/08; G07C 1/14; G01N 2035/00891; G01N 2035/00831; G01N 2035/00841; G01N 2035/00861; G06K 19/10

(57) ABSTRACT

The system comprises: a plurality of dispensing units distributed throughout the jurisdiction, each unit including: apparatus for receiving a breath sample from a person; apparatus for analysis the breath system for the presence of recreational drugs and/or alcohol; and apparatus for producing a printed or electronic ticket if the analysis of the breath sample does not reveal drugs and/or alcohol in an amount above a predetermined limit, the ticket including details of the time of the sample.

### CROSS-REFERENCE TO RELATED APPLICATION

This application claims the benefit of U.S. Provisional Patent Application Ser. No. 62/411,759, filed Oct. 24, 2016 and U.S. Provisional Patent Application Ser. No. 62/442,081, filed Jan. 4, 2017.

### BACKGROUND OF THE INVENTION

### 1. Field of the Invention

The invention relates to the field of recreational drugs and alcohol.

### 2. Prior Art

Marijuana is being legalized or decriminalized in many jurisdictions. Similarly, the sale of alcohol is being liberalized in many jurisdictions. This is causing many problems, as the institutions of many governments are not adapted to deal with this eventuality.

### SUMMARY OF THE INVENTION

Forming one aspect of the invention is a system for use in a jurisdiction, the system comprising:

a plurality of dispensing units distributed throughout the jurisdiction, each unit including:
apparatus for receiving a breath sample from a person;
apparatus for analysis the breath system for the presence of recreational drugs and/or alcohol; and
apparatus for producing a printed or electronic ticket if the analysis of the breath sample does not reveal drugs and/or alcohol in an amount above a predetermined limit, the ticket including details of the time of the sample.

Advantages features and characteristics of the invention will become apparent upon a review of the detailed description and the appended drawings, the latter being briefly described hereinafter.

### BRIEF DESCRIPTION OF THE DRAWINGS

FIG. 1 is a schematic view of an exemplary embodiment of the system; and

FIG. 2 is a view of an exemplary embodiment of encircled area 2 of FIG. 1.

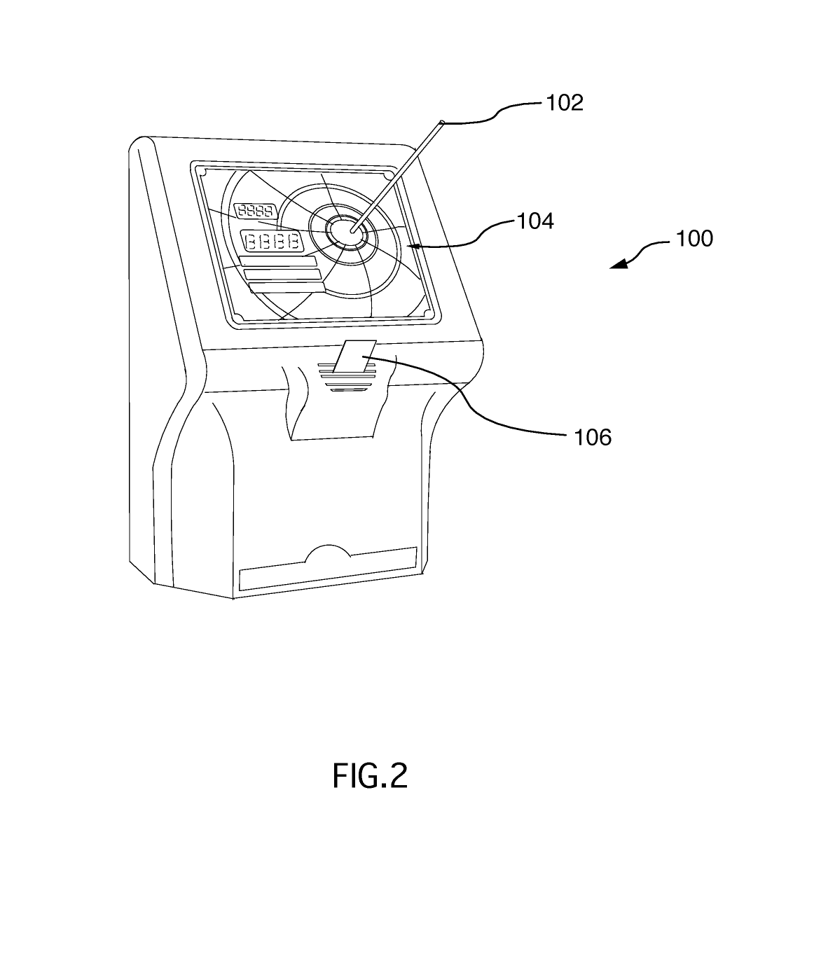

### DETAILED DESCRIPTION OF THE PREFERRED EMBODIMENTS

The embodiments discussed herein are merely illustrative of specific manners in which to make and use the invention and are not to be interpreted as limiting the scope.

While the invention has been described with a certain degree of particularity, it is to be noted that many modifications may be made in the details of the invention's construction and the arrangement of its components without departing from the scope of this disclosure. It is understood that the invention is not limited to the embodiments set forth herein for purposes of exemplification.

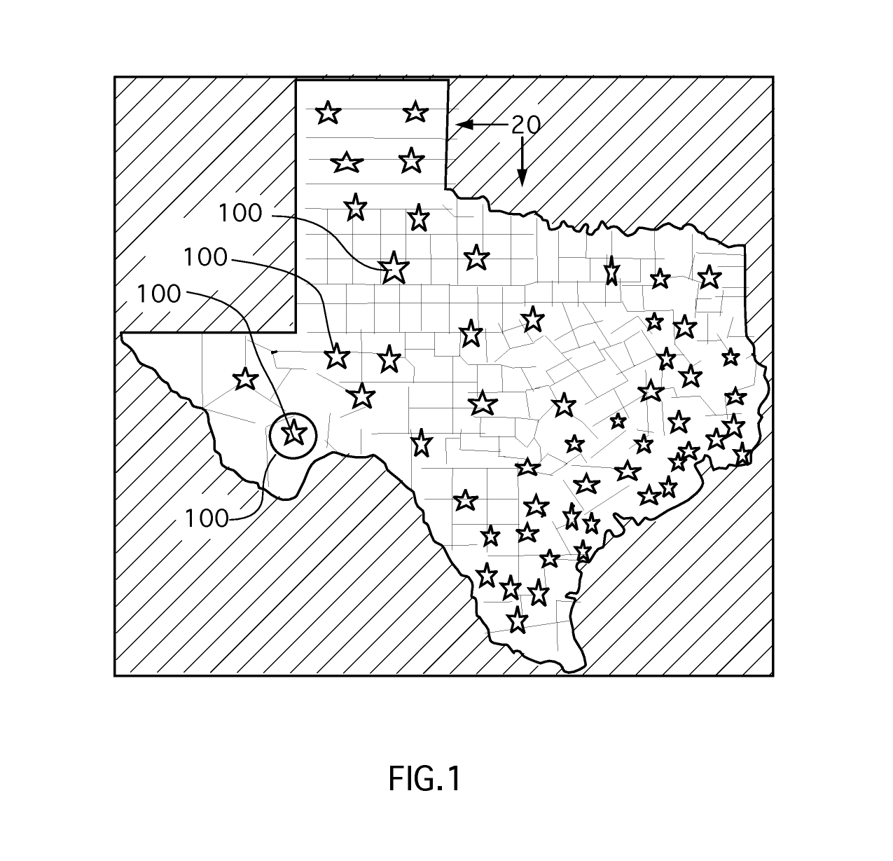

An exemplary system 20 is shown in FIG. 1 to include a plurality of dispensing units 100 distributed throughout a jurisdiction. Each unit 100 includes apparatus 102 for receiving a breath sample from a person, apparatus for testing the breath sample 104 for the presence of recreational drugs and apparatus for producing a paper or electronic ticket 106, and is in the form factor of a kiosk. The apparatus 100 shown is of the type wherein disposable straws are used by the user to provide a breath sample, to avoid contamination. The test apparatus 104 is a mass spectrometer. The ticket printer 106 will be seen to be of the paper type.

In use, persons who wish to purchase recreational drugs, such as marijuana, may attend at a dispensing unit and deliver a breath sample. If the breath sample does not reveal a high concentration of drugs, the unit produces a ticket, with a date stamp.

In a jurisdiction wherein there is no age of majority, the bearer of a ticket can attend, within a predetermined period of time following the issuance of the ticket, at an approved distribution facility to make a purchase, by presenting the ticket.

The cashier need merely visually match the time of the ticket with the current time and effect the same if the ticket was produced within a reasonable period of time. In situations wherein the apparatus is within the dispensary, then the reasonable period of time would be related to the time to walk from the apparatus to the counter. In jurisdictions where the apparatus is located centrally amongst several vendors, the reasonable time would be a function of the distance between the apparatus and the vendor who receives the ticket from the prospective purchaser.

It should be understood that the “ticket” mentioned herein may be, for example, a datum recorded on a mag strip card, a paper ticket, a data field updated on a machine readable card, or any other suitable ticket mechanism.

In a jurisdiction wherein the sale of the drug in question is restricted by age, the unit can identify the person, using a previously existing or created database, for example, a drivers licence database, which includes photographic or biometric imagery, and produce tickets only for persons of the age of majority. Alternatively, the cashier can check the ID of the prospective customer for age of majority in a conventional fashion before effecting the sale.

In the event that the system is of the type that identifies the user against a pre-existing database, the system can update a database, to reflect the fact that the user is a recreational drug user.

Some jurisdictions may elect to make an annotation on the drivers licence of the user, to make it a condition of licensure that the recreational drug user may only drive a vehicle equipped with an alcohol interlock device or equipped with means for determining the capacity of the driver, such as the Mercedes Active Lane Keeping Assist function, and which shuts down the vehicle if the driver shows evidence of impairment. If mechanisms for detecting drug impairment are developed and suitable for vehicular deployment, these would also be used.

Other jurisdictions may use the information to justify a reverse onus: persons that are recorded as recreational users may be required to deliver roadside breath samples, without probable cause, unless the vehicle which is in operation is provided with an interlock device or a Lane Keeping functionality. Jurisdictions may cross-reference the recreational user database against the vehicle ownership/insurance databases, and make such cross-reference available to peace officers involved in RIDE-type programs.

In an ideal situation, the database will be made available in a secure manner and only instantaneously, i.e. a police officer may provide to the system, by electronic communication, details of a person, and to receive, substantially instantaneously, an indication if the person is a user of the system. Further, the officer would be required to take a breath sample within a predetermined period following an affirmative indication: to avoid use by the officers for offensive purposes such as racial or socio-economic profiling.

Some jurisdictions may choose to encourage use of the dispensing units and approved distribution centres by tax measures. For example, all persons above the age of majority may be allocated and expected to pay a yearly sin tax, possibly tied to income. Persons that wish to avoid payment of the sin tax altogether could, for example, submit to periodic random blood testing, to prove that they are not recreational drug users. Other persons may be credited towards the allocated sin tax for the sin tax portion of all purchases made by the person at an authorized distribution centre.

Some jurisdictions may use the information collected for treatment purposes. For example, persons that repeatedly deliver breath samples with elevated levels of the recreational drug or alcohol to the unit may be induced to attend treatment, for example, through discounted pricing on drugs at the approved distribution centre, or modifications to the sin tax credit/allocation.

The unit could be adapted to charge a fee for each use, to offset the cost of operation.

Some jurisdictions may wish to limit the amount of information collected by the dispensing units, to encourage participation. For example, some jurisdictions may choose to collect only “clean” or “warning” levels, so as to allay fears on the part of users that the breath sample may be used for law enforcement or family law purposes.

Some jurisdictions may wish to utilize the unit for other purposes. For example, in some jurisdictions, breath testing might be made mandatory for offenders pre-adjudication and/or post sentencing, or as a condition of probation in relation to the charge or offence. In such jurisdictions, the offender could attend at the kiosk, identify biometrically and then provide a breath sample. If the sample is above a minimum threshold for alcohol or other drugs of abuse, the offender could be ordered to appear at a hearing for determination of outcome—for example, incarceration or intensive counselling.

The unit could be adapted to test for breath alcohol, only, on the grounds that alcohol and drug consumption are highly correlated and ensuring that a person buying drugs [or alcohol] is sober goes a long way, statistically, to ensuring that the same person is not impaired by drugs.

The unit could, in addition to testing for drugs and/or alcohol, test for compounds indicative of medical conditions and generally collect data useful for medical purposes. Devices such as mass spectrometers would be suitable for this purpose. The system would allow governments to collect, at the cost of, or at least subsidized by, recreational drug users, large amounts of data useful for medical research. Using units in this way would also provide an economic justification to make units available for general public diagnostic use - in a manner that does not simply add to the health care costs of the government.

In view of the foregoing, it will become apparent that modifications are possible. Accordingly, the invention should be understood to be limited only by the accompanying claims, purposively construed.

Whereas, the invention has been described in relation to the drawings attached hereto, it should be understood that other and further modifications, apart from those shown or suggested herein, may be made within the scope of this invention.

What is claimed is:

1. A system for use in a jurisdiction, the system comprising:
a plurality of dispensing units distributed throughout the jurisdiction, each unit including:
apparatus for receiving a breath sample from a person;
apparatus for analysis the breath system for the presence of recreational drugs and/or alcohol; and
apparatus for producing a printed or electronic ticket if the analysis of the breath sample does not reveal drugs and/or alcohol in an amount above a predetermined limit, the ticket being date-stamped, to enable a vendor to refuse a sale if a predetermined period of time has passed.

2. A system according to claim 1, wherein the ticket includes details of the person.

3. The system according to claim 2, wherein the apparatus includes a camera adapted to take a photograph of the person delivering the sample and the ticket includes the photograph, to enable a vendor to confirm that the bearer of the ticket is one and the same as the person that delivered the sample that resulted in the production of the ticket.

4. The system according to claim 1, wherein the predetermined period is a function of the distance between the apparatus and the vendor.

5. The system according to claim 1, wherein the apparatus
includes a reader adapted to read cards issued by the government of the jurisdiction to its residents that bear the age of the resident; and
is adapted such that, in a jurisdiction that restricts the sale of recreational drugs and/or alcohol to persons of a predetermined age, the system will not produce a ticket for a person under the predetermined age.

6. The system according to claim 1, wherein the predetermined limit is below the limit associated with impairment.

7. The system according to claim 1, wherein the apparatus is adapted such that no information is stored that could be used to prove impairment on the part of the user of the machine at the time of use.

8. The system according to claim 1, wherein the ticket is selected from the group consisting of:
paper ticket
a data entry recorded on a mag strip card
a data entry recorded on a machine readable card
a data entry recorded in a database of users

9. Use of the system of claim 8 to promote safety by:
operating the system such that persons that use the apparatus are known to the authorities; and
enforcing laws that impose reasonable restrictions upon liberty upon such persons.

10. Use according to claim 9, wherein the reasonable restrictions are selected from the group consisting of:
requiring persons that are on probation and that, as a condition of such probation, are required to refrain from the use of drugs, to periodically use the apparatus; and
modifying drivers license requirements of such persons such that they may only drive a vehicle equipped with:
an interlock device, such device requiring the delivery of a breath sample exhibiting alcohol levels below a predetermined concentration; or
functionality that determines the capacity of the driver, such as the Mercedes Active Lane Keeping Assist function, and disables the vehicle if the driver shows evidence of impairment.

11. The system according to claim 1, further comprising functionality to permit a police officer to provide to the system, by electronic communication, details of a person, and to receive, substantially instantaneously, an indication if the person is a user of the system.

12. Use of the system of claim 11 to promote safety by:
requiring an officer that has received an indication that a person is a user of the system to compel a roadside sample; and
modifying onus provisions to enable peace officers to compel a roadside sample, with no evidence of impairment or reasonable grounds, from such person.

13. Use of the system of claim 11 to promote safety by:
requiring an officer that has received an indication that a person is a user of the system to compel a roadside sample; and
modifying onus provisions to enable peace officers to compel a roadside sample, with no evidence of impairment or reasonable grounds, from such person, unless such person is driving a vehicle equipped with:
an interlock device, such device requiring the delivery of a breath sample exhibiting alcohol levels below a predetermined concentration; or
functionality that determines the capacity of the driver, such as the Mercedes Active Lane Keeping Assist function, and disables the vehicle if the driver shows evidence of impairment.
